(19) United States
(12) Patent Application Publication
(10) Pub. No.: US 2023/0285189 A1
(43) Pub. Date: Sep. 14, 2023

(54) AUTOMATIC REAL-TIME CONTROL OF ACTIVATION OF PHACOEMULSIFICATION

(71) Applicant: JOHNSON & JOHNSON SURGICAL VISION, INC., Irvine, CA (US)

(72) Inventor: Assaf Govari, Haifa (IL)

(21) Appl. No.: 17/691,321

(22) Filed: Mar. 10, 2022

(51) Int. Cl. A61F 9/007

(52) U.S. Cl. CPC A61F 9/00745; A61M 2210/0612

(57) ABSTRACT

A phacoemulsification probe includes a needle, an ultrasound transducer, a sensing element, and a microcontroller. The needle is configured for insertion into an eye of a patient. The ultrasound transducer is configured to vibrate the needle. The sensing element is configured to output an indication indicative of physical contact between the needle and a lens of the eye. The microcontroller is configured to receive the indication, and to activate and deactivate the ultrasound transducer according to the indication.

### FIELD OF THE DISCLOSURE

The present disclosure relates generally to phacoemulsification apparatuses and probes, and particularly to systems for phacoemulsification control.

### BACKGROUND OF THE DISCLOSURE

A cataract is a clouding and hardening of the eye’s natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient’s vision.

Techniques to control phacoemulsification were previously reported in the patent literature. For example, U.S. Pat. Application Publication 2005/0261628 describes a surgical system that is able to sense the onset of an occlusion or other surgical event as well as when an occlusion breaks. To help avoid overheating of the tip, the system predicts the temperature of the eye using irrigation flow rate and reduces the power to the handpiece automatically if an overheating situation is predicted. Alternatively or in addition, the system monitors the power drawn by the handpiece, which is indicative of the cutting load on the tip, and automatically adjusts the power or stroke of the tip to compensate for increased loads on the tip.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification apparatus, in accordance with an example of the present disclosure;

FIG. 2 is a block diagram, schematically describing a microcontroller-controlled activation of the phacoemulsification probe of FIG. 1, in accordance with an embodiment of the present disclosure; and

FIG. 3 is a flow chart schematically illustrating a method for automatic real-time control of activation of a phacoemulsification probe of the apparatus of FIG. 1, in accordance with an example of the present disclosure.

### DETAILED DESCRIPTION OF EXAMPLES

### Overview

During a phacoemulsification procedure, the needle of the phacoemulsification probe is vibrated with an ultrasound piezoelectric transducer. Operating the ultrasound transducer during the entire phacoemulsification procedure consumes much energy, which is both wasteful and comes with negative repercussions, such as overheating. It would be more efficient, as well as safer, to operate the ultrasound transducer only when the needle is in contact with the lens that it is emulsifying. However, it is difficult to detect when contact is established. Moreover, contact between the needle and the lens is typically intermittent. Due to the intermittency, and if the contact response time is not fast enough, the activation of the ultrasound transducer may not overlap periods of contact between the needle and the lens.

In some examples of the present disclosure that are described hereinafter a distal microcontroller (e.g., microcontroller integrated in the phacoemulsification handpiece) toggles power transmission to the ultrasound transducer in the handpiece. In other examples, the microcontroller may be integrated in a disposable unit coupled to the handpiece (e.g., to an anti-vacuum surge unit), or be located in the console.

In one example, the microcontroller activates and deactivates the ultrasound transducer according to an indication outputted by a sensing element, the indication indicative of physical contact between the needle and the lens of the eye. Specifically, the indication from a sensing element can be indicative of whether or not the needle is in contact with the lens material, in which case the microcontroller may toggle power supply to the transducer on/off according to this indication.

For example, whenever the microcontroller receives an indication in the form of a pressure reading in an aspiration channel of the probe that is below a predefined threshold, the microcontroller activates (e.g., allows power to) the ultrasound transducer to drive nominal vibration of the needle. Whenever the pressure rises above the predefined threshold the microcontroller deactivates (e.g., cuts power) to the ultrasound transducer, pausing needle operation. The drop in pressure indicates media resistance to suction due to contact with the lens. Controlling an on/off operation of the ultrasound transducer with such a distal microcontroller achieves a sufficiently fast response time (e.g., on the order of 0.1 microseconds).

The disclosed technique uses a high resolution (e.g., 32-bit) microcontroller with fast (sub-microsecond) real-time capabilities, digital signal processing, low-power/low-voltage operation, and connectivity, while maintaining a small form factor that enables the integration of such a microcontroller in the phacoemulsification handpiece, for example, the STM32 microcontroller, made by STM, which includes a DMA memory and can be used to control powering of the ultrasound transducer.

Using such a microcontroller, the phacoemulsification system avoids delays associated with the need to access the CPU and operate related software/firmware. Instead, the power line for the ultrasound transducer is connected to one of the outputs of the STM32 which may be programmed to toggle power between 0/1 (i.e., off/on) based on the current pressure in the aspiration line as compared to the predefined threshold that may be accessed with the DMA.

In other examples, the microprocessor may receive other real-time indications of needle contact with the lens that it is emulsifying, such as from an irrigation sensor that measures pressure changes indictive of pressure changes in intraocular pressure (IOP), with increased readings in irrigation fluid pressure when the needle is in contact with the lens.

An additional real-time indication of needle contact with the lens can come from an electrical change experienced by the piezoelectric crystal due to a varying mechanical load of the needle, such as manifested by the electrical impedance of the crystal. Such a change is significant for a sensing signal with a frequency that falls within the resonance line shape of the crystal. Impedance typically grows when a needle is in the lens material, and this can also provide the required real-time trigger for the microcontroller to maintain power to the crystal. When the impedance falls sufficiently, the microcontroller cuts power, though the physician maintains the option to reoperate the probe. In general, the aforementioned indication can therefore be indicative of an electrical impedance of the ultrasound transducer, and wherein the microcontroller is configured to deactivate the ultrasound transducer when the electrical impedance falls below a predefined impedance value. To this end, the transducer can be operated for very brief durations for the sake of impedance sensing (e.g., a few tens of milliseconds) to verify needle status (i.e., in the air or in the eye).

### Apparatus Description

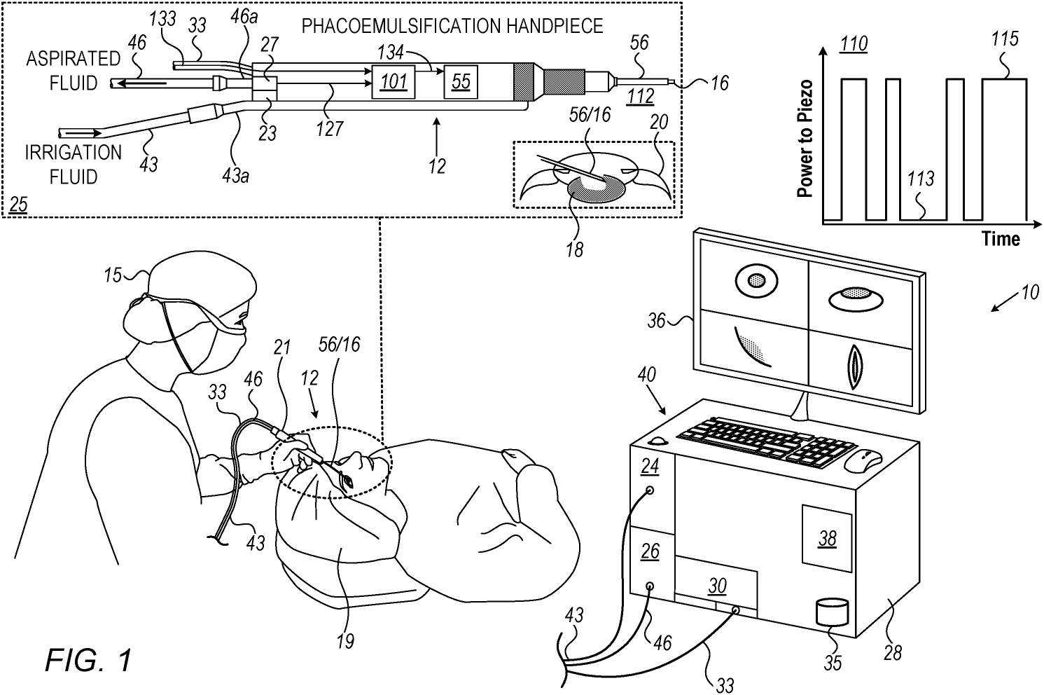

FIG. 1 is a schematic, pictorial view, along with an orthographic side view, of a phacoemulsification apparatus 10, in accordance with an example of the present disclosure.

As seen in the pictorial view of phacoemulsification apparatus 10, and in the orthographic side view inset 25, a phacoemulsification probe 12 (e.g., a handpiece) comprises a distal end 112 comprising a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the shown example, during the phacoemulsification procedure a processor-controlled irrigation pump 24 comprised in a console 28 pumps irrigation fluid from an irrigation reservoir (not shown) to the irrigation sleeve 56 to irrigate the eye. The fluid is pumped via an irrigation tubing line 43 running from console 28 to an irrigation channel 43a of probe 12. In another example, pump 24 may be coupled with or replaced by a gravity-fed irrigation source such as a balanced salt solution bottle/bag. In general, irrigation plump 24 may be located outside the console.

Eye fluid and waste matter (e.g., emulsified parts of the cataract) are aspirated via hollow needle 16 to a collection receptacle (not shown) by a processor-controlled aspiration pump 26, also comprised in console 28, using aspiration tubing line 46 running from aspiration channel 46a of probe 12 to console 28. In an example, processor 38 controls an aspiration rate of aspiration pump 26 to maintain intraocular pressure (e.g., in case of sub-pressure indicated, for example, by sensor 27) within prespecified limits.

Channels 43a and 46a are coupled with irrigation line 43 and aspiration line 46, respectively. Pumps 24 and 26 may be any pump known in the art (e.g., a peristaltic pump). Using sensors (e.g., as indicated by sensors 23 and/or 27), processor 38 controls a pump rate of irrigation pump 24 and aspiration pump 26 to maintain intraocular pressure (IOP) within prespecified limits. In general, aspiration plump 26 may be located outside the console.

In the shown example, probe 12 includes an irrigation sensor 23 coupled with irrigation channel 43a and an aspiration sensor 27 coupled with an aspiration channel 46a. Sensors 23 and 27 may be any sensor known in the art, including, but not limited to, a vacuum sensor or flow sensor. The sensor measurements (e.g., pressure, vacuum, and/or flow) may be taken close to the proximal end of the handpiece where the irrigation outlet and the aspiration inlet are located, so as to provide processor 38 with an accurate indication of the actual measurements occurring within an eye and provide a short response time to a control loop comprised in a microcontroller 101 comprised in probe 12, as described below.

A vacuum sensor, as discussed herein, includes types of pressure sensors that are configured to provide sufficiently accurate measurements of low sub-atmospheric pressures within a typical sub-pressure range at which aspiration is applied (e.g., between 1 mmHg and 650 mmHg). In an example, the same pressure sensor model is used to measure irrigation pressure and aspiration sub-pressure, using different sensor settings/calibrations.

As further shown, phacoemulsification probe 12 includes one or more piezoelectric crystals (or ultrasound transducer) 55, coupled with a horn (not shown), that drives needle 16 to vibrate in a resonant vibration mode that is used to break a cataract into small pieces during a phacoemulsification procedure. Console 28 comprises a piezoelectric drive module 30 that is controlled by a processor 38, which conveys processor-controlled driving signals via wiring 133 to, for example, maintain needle 16 at maximal vibration amplitude. The drive module may be realized in hardware or software, for example, in a proportional-integral-derivative (PID) control architecture.

As seen, drive module 30 is coupled with a power port of microcontroller 101 using electrical wiring 133 running in cable 33. Microcontroller 101 has an output port with wiring 134 to piezoelectric crystal(s) 55. Using readings received via wiring 127 from sensor 27, microcontroller 101 modulates the power, in real time, to piezoelectric crystal 55. An example of the powering of needle 16 with piezoelectric crystal 55 is given in graph 110. As seen, crystal power varies between zero power (113), with no needle vibration, to nominal power (115) with nominal needle vibration. Periods of time with zero power 113 correspond to sensor 27 readings indictive of needle 16 not in contact with lens 18 material, while periods of time with nominal power 115 correspond to sensor 27 readings indictive of needle 16 in contact with lens 18 material, as described above.

Processor 38 may receive user-based commands via a user interface 40, which may include setting a vibration mode and/or frequency of the piezoelectric crystal, and setting or adjusting an irrigation and/or aspiration rate of the irrigation pump 24 and aspiration pump 26. Processor 38 may receive user-based commands via a user interface 40, which may include needle 16 stroke amplitude settings and activating irrigation and/or aspiration. In an example, the physician uses a foot pedal (not shown) as a means of control, where pedal position one activates irrigation only, pedal position two activates both irrigation and aspiration, and pedal position three adds needle 16 vibration. Additionally, or alternatively, processor 38 may receive user-based commands from controls located in a handle 21 of probe 12.

In an example, user interface 40 and display 36 may be integrated into a touch screen graphical user interface.

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 may include further elements, which are omitted for clarity of presentation. For example, physician 15 typically performs the procedure using a stereo microscope or magnifying glasses, neither of which is shown. Physician 15 may use other surgical tools in addition to probe 12, which are also not shown in order to maintain clarity and simplicity of presentation.

### Automatic Real-time Control of Phacoemulsification Activation

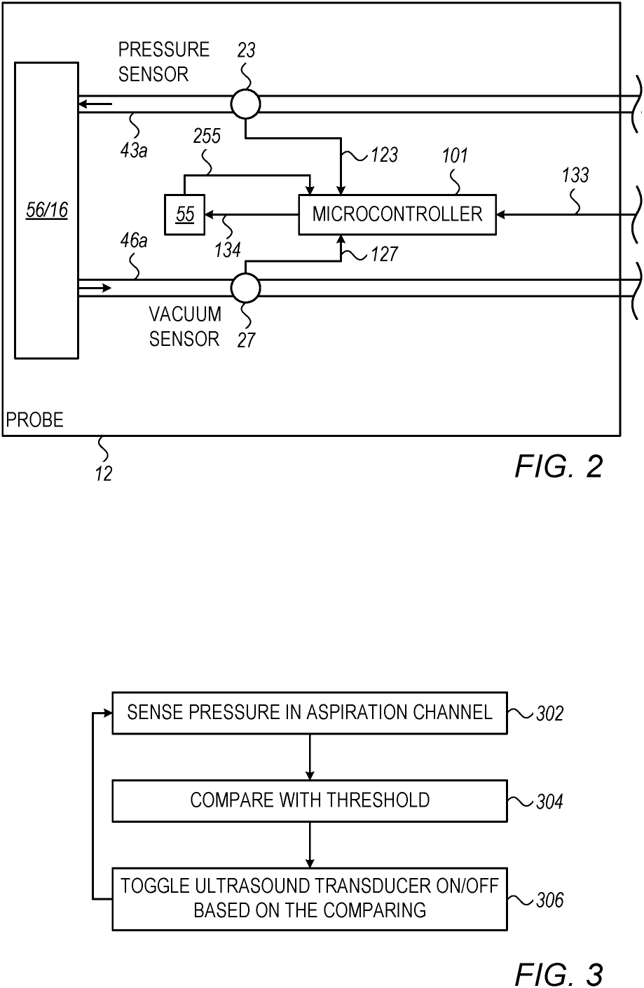

FIG. 2 is a block diagram schematically describing a microcontroller-controlled activation of phacoemulsification probe 12 of FIG. 1, in accordance with some examples of the present disclosure.

As seen, microcontroller 101 of probe 12 receives readings via wiring 123 and/or 127 from respective sensors 23 and/or 27 (e.g., pressure, vacuum, or flow) of irrigation channel 43a, and of aspiration channel 46a, respectively. The readings are provided via wiring 123 and/or 127. These are provided (e.g., sampled) in real time at a sufficiently high rate (e.g., 1 kHz) to allow a fast system response time (e.g., within several milliseconds). In the shown example, sensors 23 and 27 are seen as located in handpiece 12. In general, the sensors can be located in a case or module coupled with the handpiece, e.g., by including the sensors in a disposable case coupled to the aspiration and irrigation lines just proximally of the handle itself.

In response to readings from sensors 23 and/or 27, microcontroller 101, which is fed with power via wiring 133, toggles between power on and power off to piezoelectric crystal 55 via wiring 134. Alternatively, the toggling can be between nominal power and very low power. As noted above, in one example, the microcontroller is configured to channel power to the piezoelectric crystal 55 whenever the pressure reading in aspiration channel 46a is below a predefined threshold, and to cut power to piezoelectric crystal 55 whenever the pressure rises above the predefined threshold. In another example, the microcontroller is configured to channel power to the ultrasound transducer whenever the pressure reading in the irrigation channel is above a predefined threshold, and cut power to the ultrasound transducer whenever the pressure falls below the predefined threshold for irrigation pressure.

In yet another example, the microcontroller is configured to channel power to the piezoelectric crystal 55 whenever the vacuum level in aspiration channel 46a is above a predefined threshold, and to cut power to piezoelectric crystal 55 whenever the vacuum level falls below the predefined threshold (i.e., there is not enough vacuum or suction force).

In a further example, the microcontroller is configured to channel power to the piezoelectric crystal 55 whenever the sensed flow rate in aspiration channel 46a is below a predefined threshold, and to cut power to piezoelectric crystal 55 whenever the flow rate is above the predefined threshold.

Additionally or alternatively, microcontroller 101 may receive impedance readings via wiring 255 from ultrasound piezoelectric crystal 55. To this end, microcontroller 101 can generate, or channel, a small-amplitude monitoring signal (e.g., at the drive frequency at a detuned frequency). A small amplitude signal has a frequency that falls into the line shape of the mechanical frequency (e.g., a few percentage points detuned from the resonance frequency). A sensing circuitry in microcontroller 101 measures the applied voltage and the current of the sensing signal and determines the changed impedance of electrical impedance of piezoelectric crystal 55 from the voltage and current.

When the needle is touching the lens material, the impedance typically grows, and this can also provide the required real-time trigger for the microcontroller to maintain power to the crystal. When the impedance falls sufficiently, the microcontroller cuts power, though the physician has the option to reoperate the probe. The transducer can also be operated for very brief durations (e.g., a few tens of milliseconds) of impedance sensing to verify status of needle (in the air or in the eye).

The example block diagram shown in FIG. 2 is highly simplified and was chosen purely for the sake of conceptual clarity. A typical system may include, for example, a more complex wiring scheme.

FIG. 3 is a flow chart schematically illustrating a method for automatic real-time control of activation of a phacoemulsification probe of the apparatus of FIG. 1, in accordance with an example of the present disclosure.

The process assumes that physician 15 is inserting phacoemulsification needle 16 of probe 12 into a lens capsule 18 of an eye 20, presses a foot pedal (not shown) to a first position to activate irrigation, subsequently to a second position to activate aspiration, and finally, when the foot pedal is pressed and placed in a third position, the needle 16 is vibrated to perform phacoemulsification.

In the shown process, at aspiration pressure sensing receiving step 302, microcontroller 101 receives repeated aspiration pressure readings from sensor 27 (e.g., at a 1 KHz rate) during phacoemulsification.

Microcontroller 101 compares the aspiration pressure readings to a given threshold, at a comparing step 304.

In response to the comparison, microcontroller 101 toggles power on/off to piezoelectric crystal 55, depending on whether the needle is indicated by the aspiration pressure to be touching or not touching lens material, at a power toggling step 306.

The steps of FIG. 3 are brought as an example. As another example, instead of using readings from sensor 27, microcontroller 101 can use pressure readings from sensor 23, and/or use impedance readings of piezoelectric crystal 55, as described above.

### EXAMPLES

### Example 1

A phacoemulsification probe (12) includes a needle (16), an ultrasound transducer (55), a sensing element (23, 27, 55), and a microcontroller (101). The needle is configured for insertion into an eye (20) of a patient. The ultrasound transducer is configured to vibrate the needle. The sensing element is configured to output an indication indicative of physical contact between the needle and a lens (18) of the eye. The microcontroller is configured to receive the indication, and to activate and deactivate the ultrasound transducer according to the indication.

### Example 2

The probe according to example 1, wherein the sensing element is an aspiration pressure sensor (27) coupled with an aspiration channel (46a) comprised in the probe (12), and wherein the indication is indicative of a pressure in the aspiration channel.

### Example 3

The probe according to a example 2, wherein the microcontroller (101) is configured to activate the ultrasound transducer (55) when the pressure in the aspiration channel (46a) is below a predefined threshold, and to deactivate the ultrasound transducer when the pressure is above the predefined threshold.

### Example 4

The probe according to example 1, wherein the sensing element is vacuum sensor coupled with an aspiration channel (46a) comprised in the probe (12), and wherein the indication is indicative of a vacuum level in the aspiration channel.

### Example 5

The probe according to example 4, wherein the microcontroller (101) is configured to activate the ultrasound transducer (55) when the vacuum in the aspiration channel is above a predefined threshold, and to deactivate the ultrasound transducer when the vacuum level is below the predefined threshold.

### Example 6

The probe according to example 1, wherein the sensing element is an aspiration flow rate sensor coupled with an aspiration channel (46a) comprised in the probe (12), and wherein the indication is indicative of a flow rate in the aspiration channel.

### Example 7

The probe according to example 6, wherein the microcontroller (101) is configured to activate the ultrasound transducer (55) when the flow rate in the aspiration channel (46a) is below a predefined threshold, and to deactivate the ultrasound transducer when the flow rate is above the predefined threshold.

### Example 8

The probe according to example 1, wherein the sensing element is an irrigation pressure sensor coupled with an irrigation channel (43a) comprised in the probe (12), and wherein the indication is indicative of a pressure in the irrigation channel.

### Example 9

The probe according to example 8, wherein the microcontroller (101) is configured to activate the ultrasound transducer (55) when the pressure in the irrigation channel (43a) is above a predefined threshold, and deactivate the ultrasound transducer when the pressure is below the predefined threshold.

### Example 10

The probe (12) according to example 1, wherein the indication is indicative of an electrical impedance of the ultrasound transducer (55), and wherein the microcontroller (101) is configured to deactivate the ultrasound transducer (55) when the electrical impedance falls below a predefined impedance value.

### Example 11

The probe (12) according to example 1, wherein the microcontroller (101) is configured to deactivate the ultrasound transducer (55) by cutting power to the ultrasound transducer.

### Example 12

A phacoemulsification method includes inserting a needle (16) into an eye (20) of a patient. The needle is vibrated using an ultrasound transducer (55). An indication is outputted, indicative of physical contact between the needle (16) and a lens (18) of the eye (20) using a sensing element (23, 27, 55). The indication is received in a microcontroller (101), and the ultrasound transducer (55) is activated and deactivated according to the indication.

Although the embodiments described herein mainly address a phacoemulsification technique, the methods and systems described herein can also be used in other applications, such as with other types of electrical eye surgery tools.

It will be thus appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A phacoemulsification probe, comprising:
a needle configured for insertion into an eye of a patient;
an ultrasound transducer configured to vibrate the needle;
a sensing element configured to output an indication indicative of physical contact between the needle and a lens of the eye; and
a microcontroller, which is configured to receive the indication, and to activate and deactivate the ultrasound transducer according to the indication.

2. The probe according to claim 1, wherein the sensing element is an aspiration pressure sensor coupled with an aspiration channel comprised in the probe, and wherein the indication is indicative of a pressure in the aspiration channel.

3. The probe according to claim 2, wherein the microcontroller is configured to activate the ultrasound transducer when the pressure in the aspiration channel is below a predefined threshold, and to deactivate the ultrasound transducer when the pressure is above the predefined threshold.

4. The probe according to claim 1, wherein the sensing element is vacuum sensor coupled with an aspiration channel comprised in the probe, and wherein the indication is indicative of a vacuum level in the aspiration channel.

5. The probe according to claim 4, wherein the microcontroller is configured to activate the ultrasound transducer when the vacuum in the aspiration channel is above a predefined threshold, and to deactivate the ultrasound transducer when the vacuum level is below the predefined threshold.

6. The probe according to claim 1, wherein the sensing element is an aspiration flow rate sensor coupled with an aspiration channel comprised in the probe, and wherein the indication is indicative of a flow rate in the aspiration channel.

7. The probe according to claim 6, wherein the microcontroller is configured to activate the ultrasound transducer when the flow rate in the aspiration channel is below a predefined threshold, and to deactivate the ultrasound transducer when the flow rate is above the predefined threshold.

8. The probe according to claim 1, wherein the sensing element is an irrigation pressure sensor coupled with an irrigation channel comprised in the probe, and wherein the indication is indicative of a pressure in the irrigation channel.

9. The probe according to claim 8, wherein the microcontroller is configured to activate the ultrasound transducer when the pressure in the irrigation channel is above a predefined threshold, and deactivate the ultrasound transducer when the pressure is below the predefined threshold.

10. The probe according to claim 1, wherein the indication is indicative of an electrical impedance of the ultrasound transducer, and wherein the microcontroller is configured to deactivate the ultrasound transducer when the electrical impedance falls below a predefined impedance value.

11. The probe according to claim 1, wherein the microcontroller is configured to deactivate the ultrasound transducer by cutting power to the ultrasound transducer.

12. A phacoemulsification method, comprising:
inserting a needle into an eye of a patient;
vibrating the needle using an ultrasound transducer;
outputting an indication indicative of physical contact between the needle and a lens of the eye using a sensing element; and
receiving the indication in a microcontroller, and activating and deactivating the ultrasound transducer according to the indication.

13. The method according to claim 12, wherein the sensing element is an aspiration pressure sensor coupled with an aspiration channel comprised in the probe, and wherein the indication is indicative of a pressure in the aspiration channel.

14. The method according to claim 13, wherein the microcontroller is configured to activate the ultrasound transducer when the pressure in the aspiration channel is below a predefined threshold, and to deactivate the ultrasound transducer when the pressure is above the predefined threshold.

15. The method according to claim 12, wherein the sensing element is vacuum sensor coupled with an aspiration channel comprised in the probe, and wherein the indication is indicative of a vacuum level in the aspiration channel.

16. The method according to claim 15, wherein the microcontroller is configured to activate the ultrasound transducer when the vacuum in the aspiration channel is above a predefined threshold, and to deactivate the ultrasound transducer when the vacuum level is below the predefined threshold.

17. The method according to claim 12, wherein the sensing element is an aspiration flow rate sensor coupled with an aspiration channel comprised in the probe, and wherein the indication is indicative of a flow rate in the aspiration channel.

18. The method according to claim 17, wherein the microcontroller is configured to activate the ultrasound transducer when the flow rate in the aspiration channel is below a predefined threshold, and to deactivate the ultrasound transducer when the flow rate is above the predefined threshold.

19. The method according to claim 12, wherein the sensing element is an irrigation pressure sensor coupled with an irrigation channel comprised in the probe, and wherein the indication is indicative of a pressure in the irrigation channel.

20. The method according to claim 19, wherein the microcontroller is configured to activate the ultrasound transducer when the pressure in the irrigation channel is above a predefined threshold, and deactivate the ultrasound transducer when the pressure is below the predefined threshold.

21. The method according to claim 12, wherein the indication is indicative of an electrical impedance of the ultrasound transducer, and wherein the microcontroller is configured to deactivate the ultrasound transducer when the electrical impedance falls below a predefined impedance value.

22. The method according to claim 12, wherein the microcontroller is configured to deactivate the ultrasound transducer by cutting power to the ultrasound transducer.
